(19) United States
(12) Patent Application Publication
(10) Pub. No.: US 2021/0015850 A1
(43) Pub. Date: Jan. 21, 2021

(54) COMPOSITION FOR IMPROVING INTESTINAL BARRIER FUNCTION

(71) Applicant: SUNTORY HOLDINGS LIMITED, Osaka-shi, Osaka (JP)

(72) Inventors: Ayuta Funaki, Soraku-gun (JP); Naoki Kasajima, Soraku-gun (JP); Hideyuki Arie, Soraku-gun (JP)

(73) Assignee: SUNTORY HOLDINGS LIMITED, Osaka-shi, Osaka (JP)

(21) Appl. No.: 16/957,583

(22) PCT Filed: Dec. 26, 2018
(86) PCT No.: PCT/JP2018/047877

§ 371 Date: Jun. 24, 2020

(30) Foreign Application Priority Data
Dec. 27, 2017 (JP) 2017-251994

(51) Int. Cl. A61K 31/765; A61K 31/192; A61P 1/14

(52) U.S. Cl. CPC A61K 31/765; A61P 1/14; A61K 31/192

(57) ABSTRACT

The present invention relates to a composition for improving intestinal barrier function, the composition containing a compound having a gallic acid residue as an active ingredient, wherein the compound having a gallic acid residue is at least one compound selected from the group consisting of the following (A1) to (A3): (A1) a flavan-3-ol polymer having a galloyl group; (A2) a hydrolyzable tannin; and (A3) at least one compound selected from the group consisting of catechin gallate, epicatechin gallate, and gallocatechin gallate.

### TECHNICAL FIELD

The present invention relates to a composition for improving intestinal barrier function. The present invention also relates to a method for improving intestinal barrier function, and use of a compound having a gallic acid residue for improving intestinal barrier function.

### BACKGROUND ART

In recent years, awareness with regard to intestinal health has increased, and a large number of intestine-related functional foods have been also sold. An intestinal function mainly includes a nutrient absorption function and a barrier function (intestinal barrier function) which prevents the intrusion (permeation) of toxic substances. Among these, it has become clear that the intestinal barrier function is deeply involved with chronic inflammation diseases increasing with aging.

Underneath intestinal epithelial cells, there exist a great number of immune cells such as macrophages, dendritic cells, T-cells, and B-cells. Typically, the intestinal epithelial cells are tightly joined to each other by a structure referred to as a tight junction, so that high molecular weight substances are strictly controlled so as not to pass through intercellular spaces. The intestinal epithelial cells contain transporters for removing hydrophobic foreign substances from the cells. The tight junction structure, the transporters and the like are responsible for the intestinal barrier function which prevents the intrusion of foreign substances. However, when the intestinal barrier is damaged by aging, neglect of health in life, stress and the like, leading to an increase in intestinal permeability, high molecular weight substances such as intestinal bacteria and their bacterial ingredients, which are present in the intestine, penetrate through intercellular spaces into the body and stimulate immune cells to release inflammatory cytokine, thereby inducing inflammation. As a result, various disease states caused by chronic inflammation such as inflammatory bowel diseases in the intestine, non-alcoholic fatty liver diseases (NAFLD) in the liver, arteriosclerosis-induced symptoms in the blood vessel, generalized diabetes, abnormal lipid metabolism, and autoimmune diseases are considered to be induced. An undigested substance is assumed to intrude out of the intestine to induce allergies.

As described above, the reduction of the intestinal barrier function may cause various diseases or the like. From such a viewpoint, searches for materials which can improve the intestinal barrier function have been attempted. An epithelial cell growth factor (EGF) has been known to promote the maturation of the intestinal epithelial cells, to enhance a barrier function. However, only a small amount of the EGF which is cytokine exists in the living body, so that it is not preferable to use the EGF as a material which improves the intestinal barrier function in respects of economic efficiency and safety. Non-Patent Literature 1 discloses that flavonoid such as quercetin promotes the formation of a tight junction or the like to prevent chronic inflammation. Patent Literature 1 discloses an absorption depressant which contains one or two or more selected from lindane, star anise, marnie, tea, black tea, or treated products thereof as active ingredient(s). Patent Literature 2 discloses that hexapeptide of a specific sequence and tryptophan have absorption suppressive activity of allergen. Patent Literature 3 discloses a supplement to be administered enterally to maintain or restore the intestinal barrier of the intestine, including a combination of glutamine, a substance having antioxidant activity, and a short-chain fatty acid.

### CITATION LIST

### Patent Literature

Patent Literature 1: JP 2002-193819 A
Patent Literature 2: JP 2002-257814 A
Patent Literature 3: JP 2004-513912 T

### Non-Patent Literature

Non-Patent literature 1: Suzuki T. et al, The Journal of Nutritional Biochemistry. 2011 May, Vol. 22(5), p. 401-408

### SUMMARY OF INVENTION

### Technical Problem

An object of the present invention is to provide a composition for improving intestinal barrier function, the composition capable of improving intestinal barrier function.

### Solution to Problem

In order to solve the above problems, the present inventors intensively studied, and tried to solve the problems by adding inflammatory cytokine to an intestinal permeation model using human intestinal cell cultivated strain Caco-2, to produce a state where intestinal barrier function can be collapsed in humans, and finding a substance capable of ameliorating the state. As a result, the present inventors found that a compound having a gallic acid residue such as a flavan-3-ol polymer having a galloyl group is useful for improving the intestinal barrier function.

That is, the present invention relates to the following composition for improving intestinal barrier function.

(1) A composition for improving intestinal barrier function, the composition containing: a compound having a gallic acid residue as an active ingredient, wherein the compound having a gallic acid residue is at least one compound selected from the group consisting of the following (A1) to (A3):

(A1) a flavan-3-ol polymer having a galloyl group;

(A2) a hydrolyzable tannin; and

(A3) at least one compound selected from the group consisting of catechin gallate, epicatechin gallate, and gallocatechin gallate.

(2) The composition for improving intestinal barrier function according to the above (1), wherein the compound having a gallic acid residue is the flavan-3-ol polymer having a galloyl group.

(3) The composition for improving intestinal barrier function according to the above (1) or (2), wherein the flavan-3-ol polymer having a galloyl group is contained in at least one grape-derived raw material selected from the group consisting of grape pulp, grape seed coat, and grape seed.

(4) The composition for improving intestinal barrier function according to any one of the above (1) to (3), wherein an amount of gallic acid produced by tannase treatment is 0.1 μg or more per mg of the flavan-3-ol polymer having a galloyl group.

(5) The composition for improving intestinal barrier function according to the above (1), wherein the hydrolyzable tannin is gallotannin or ellagitannin.

(6) The composition for improving intestinal barrier function according to the above (5), wherein the gallotannin is a compound in which 3 to 5 molecules of gallic acid are bound to one molecule of glucose.

(7) The composition for improving intestinal barrier function according to the above (5) or (6), wherein the gallotannin is at least one compound selected from the group consisting of 1,4,6-tri-O-galloyl-β-D-glucose, 1,2,4,6-tetra-O-galloyl-β-D-glucose, 1,2,3,6-tetra-O-galloyl-β-D-glucose, 2,3,4,6-tetra-O-galloyl-β-D-glucose, and 1,2,3,4,6-penta-O-galloyl-β-D-glucose.

(8) The composition for improving intestinal barrier function according to the above (5), wherein the ellagitannin is at least one compound selected from the group consisting of tellimagrandin I, pedunculagin, praecoxin A, geraniin, stenophyllanin A, stenophyllanin B, casuarinin, and eugeniflorin D2.

(9) The composition for improving intestinal barrier function according to any one of the above (1) to (8), wherein the composition for improving intestinal barrier function is an oral composition.

(10) The composition for improving intestinal barrier function according to the above (9), wherein the oral composition is a food or beverage, a pharmaceutical product, or a quasi-pharmaceutical product.

(11) The composition for improving intestinal barrier function according to any one of the above (1) to (10), wherein the composition is used for intestinal regulation by improving the intestinal barrier function.

(12) The composition for improving intestinal barrier function according to any one of the above (1) to (11), wherein the composition is used in order to prevent or relieve abdominal discomfort by improving the intestinal barrier function.

(13) The composition for improving intestinal barrier function according to any one of the above (1) to (12), wherein the composition is labeled as having an intestinal regulation action.

(14) A method for improving intestinal barrier function, the method including: administering at least one compound having a gallic acid residue selected from the group consisting of the following (A1) to (A3) to a subject:

(A1) a flavan-3-ol polymer having a galloyl group;

(A2) a hydrolyzable tannin; and

(A3) at least one compound selected from the group consisting of catechin gallate, epicatechin gallate, and gallocatechin gallate.

(15) Use of at least one compound having a gallic acid residue selected from the group consisting of the following (A1) to (A3) for improving intestinal barrier function:

(A1) a flavan-3-ol polymer having a galloyl group;

(A2) a hydrolyzable tannin; and

(A3) at least one compound selected from the group consisting of catechin gallate, epicatechin gallate, and gallocatechin gallate.

### Advantageous Effects of Invention

The use of a composition for improving intestinal barrier function according to the present invention makes it possible to improve intestinal barrier function. The present invention improves the intestinal barrier function, which makes it possible to contribute also to prevention or amelioration of conditions or diseases related to intestinal barrier dysfunction.

### BRIEF DESCRIPTION OF DRAWINGS

FIG. 1 is a flow chart showing a procedure of purifying a flavan-3-ol polymer (OPC) having a galloyl group from a grape seed extract.

FIG. 2 shows a schedule for a test of Example 6.

FIG. 3 shows graphs of results of examining the effect of a flavan-3-ol polymer having a galloyl group on the pain threshold of the large intestine, the intestinal permeability of the large intestine, and the expression of Claudin-2 ((a) pain threshold, (b) intestinal permeability of the large intestine, (c) Claudin-2 relative expression level).

### DESCRIPTION OF EMBODIMENTS

A composition for improving intestinal barrier function according to the present invention contains a compound having a gallic acid residue as an active ingredient, wherein the compound having a gallic acid residue is at least one compound selected from the group consisting of the following (A1) to (A3):

(A1) a flavan-3-ol polymer having a galloyl group;

(A2) a hydrolyzable tannin; and

(A3) at least one compound selected from the group consisting of catechin gallate, epicatechin gallate, and gallocatechin gallate.

The composition for improving intestinal barrier function according to the present invention may contain only one of the compounds selected from the group consisting of the above (A1) to (A3), or may contain two or more of the compounds.

In the present invention, the gallic acid residue refers to a galloyl group or a residue (gallic acid oligomer residue) formed from a gallic acid oligomer. The gallic acid oligomer is a gallic acid dimer to tetramer in which gallic acids (3,4,5-trihydroxybenzoic acid) bind via ether bonds, ester bonds, carbon-carbon bonds or the like, preferably a gallic acid dimer or trimer, more preferably a gallic acid dimer. Examples of the residue of the gallic acid dimer include a hexahydroxydiphenoyl group (HHDP group). Examples of the residue of the gallic acid trimer include a valoneoyl group (a group in which gallic acid is added to an HHDP group). In one aspect, the compound having a gallic acid residue in the present invention is preferably a compound having at least one gallic acid residue selected from the group consisting of a galloyl group, a residue of a gallic acid dimer, and a residue of a gallic acid trimer, more preferably a compound having a galloyl group and/or a residue of a gallic acid dimer.

Each of the compounds (A1) to (A3) has a gallic acid residue in its molecule. Each of the compounds (A1) to (A3) is an ingredient contained in a plant such as grape, and has few side effects and high safety even if the compound is ingested for a long period of time. The present invention can provide the composition for improving intestinal barrier function, the composition containing a substance having few side effects and high safety as an active ingredient even if the composition is ingested for a long period of time.

In one aspect, the compound having a gallic acid residue in the present invention is preferably the flavan-3-ol polymer having a galloyl group (A1) and/or the hydrolyzable tannin (A2), more preferably the flavan-3-ol polymer having a galloyl group (A1).

The flavan-3-ol polymer in the present invention contains flavan-3-ol as a structural unit, and is a dimer or higher polymer with 4-6 or 4-8 interflavan bonds formed by condensation or polymerization. Examples of the flavan-3-ol include catechin and epicatechin. The flavan-3-ol polymer is one of polyphenols, and is a compound referred to as condensed tannin.

The flavan-3-ol polymer having a galloyl group (A1) is a polymer in which at least one structural unit thereof is flavan-3-ol having a galloyl group. The binding position of the galloyl group in the flavan-3-ol as the structural unit is not limited, and for example, the galloyl group may bind to a flavan skeleton. In one aspect, the flavan-3-ol polymer having a galloyl group in the present invention may be a flavan-3-ol polymer containing, as a structural unit, flavan-3-ol in which a galloyl group binds to position 3 of a flavan skeleton (flavan-3-ol in which a hydroxyl group at position 3 of the flavan skeleton is esterified to a carboxyl group of gallic acid).

The flavan-3-ol polymer having a galloyl group may be a mixture of two or more polymers having different degrees of polymerization or the like. The flavan-3-ol polymer having a galloyl group may have a gallic acid residue besides the galloyl group.

The flavan-3-ol polymer having a galloyl group is not limited by the derivation or the producing method. For example, a plant-derived flavan-3-ol polymer extracted from a plant may be used, and a flavan-3-ol polymer obtained by a synthetic method may be used. For example, the flavan-3-ol polymer having a galloyl group can be obtained from plants such as grape and pyrus communis. In terms of the intestinal barrier function improvement effect, the flavan-3-ol polymer having a galloyl group is preferably one derived from grape. More preferably, the flavan-3-ol polymer is at least one selected from the group consisting of one contained in (derived from) grape pulp, one contained in (derived from) grape seed coat, and one contained in (derived from) grape seed. Still more preferably, the flavan-3-ol polymer is derived from grape seed (for example, derived from a grape seed extract).

For example, when the flavan-3-ol polymer is extracted and purified from the grape seed, the grape seed is extracted by hydrous alcohol, and the obtained extract is then filtered and the alcohol is removed, followed by performing column purification, whereby the grape seed extract containing the flavan-3-ol polymer having a galloyl group can be obtained. The obtained grape seed extract is further purified, whereby the purity of the flavan-3-ol polymer having a galloyl group can also be increased.

For example, when the flavan-3-ol polymer is subjected to tannase treatment or acid hydrolysis treatment, gallic acid is produced, whereby the flavan-3-ol polymer having the galloyl group can be confirmed. The flavan-3-ol polymer producing gallic acid by the treatment has the galloyl group. The flavan-3-ol polymer having a galloyl group (A1) in the present invention may also be referred to as flavan-3-ol polymer producing gallic acid by the tannase treatment or the acid hydrolysis treatment (in one aspect, preferably, the tannase treatment).

The production of gallic acid can be confirmed by measuring the amounts of gallic acid before and after the tannase treatment or the acid hydrolysis treatment for the flavan-3-ol polymer. When the amount of gallic acid in the flavan-3-ol polymer after the treatment is more than the amount of gallic acid in the flavan-3-ol polymer before the treatment, gallic acid can be deemed to be produced by the above treatment.

The acid hydrolysis treatment can be performed by, for example, the following method. A sample powder is dissolved at 2 mg/mL in 20% acetonitrile, and to the resulting solution, an equal amount of 6N hydrochloric acid is added. The solution is heated at 100° C. for 3 hours, and left to stand until the solution reaches room temperature. The reaction solution reaching room temperature can be used for measurement of the amount of gallic acid as the sample after the acid hydrolysis treatment.

The tannase treatment means that a compound having a gallic acid residue such as a flavan-3-ol polymer and an enzyme having tannase (tannin acyl hydrolase) activity are reacted. The enzyme having tannase activity is preferably, for example, tannase derived from Aspergillus oryzae. The tannase treatment can be performed under conditions of Examples to be described later.

In the flavan-3-ol polymer having a galloyl group, the amount of gallic acid produced by the tannase treatment is preferably 0.1 μg or more, more preferably 1 μg or more, still more preferably 10 μg or more per mg of the flavan-3-ol polymer having a galloyl group. If the flavan-3-ol polymer in which the amount of gallic acid produced by the tannase treatment is within the above range is used, a higher intestinal barrier function improvement effect is obtained. The upper limit of the amount of gallic acid produced by the tannase treatment is not limited for the flavan-3-ol polymer having a galloyl group. In one aspect, in the flavan-3-ol polymer having a galloyl group, the amount of gallic acid produced by the tannase treatment may be, for example, 100 μg or less or 50 μg or less per mg of the flavan-3-ol polymer having a galloyl group.

In one aspect, in the flavan-3-ol polymer having a galloyl group, the amount of gallic acid produced by the tannase treatment is preferably 0.1 to 100 μg, more preferably 1 to 100 μg, still more preferably 10 to 100 μg, particularly preferably 10 to 50 μg per mg of the flavan-3-ol polymer having a galloyl group.

In the measurement of the amount of gallic acid produced by the tannase treatment, the tannase treatment can be performed under conditions described in Examples. The amount of gallic acid can be measured by an external standard method using HPLC under conditions described in Examples.

The amount of gallic acid produced by the tannase treatment is determined according to the following calculating formula from the amounts of gallic acid in the flavan-3-ol polymer before the tannase treatment (the sample before the treatment) and the flavan-3-ol polymer after the tannase treatment (the sample after the treatment).

Amount of gallic acid produced by tannase treatment=(Amount of gallic acid in sample after treatment)−(Amount of gallic acid in sample before treatment)

The amount of gallic acid produced by the tannase treatment can be regarded as the amount of the galloyl group in terms of the weight of gallic acid produced by the tannase treatment. In the flavan-3-ol polymer having a galloyl group, the content of the galloyl group is preferably within the above range in terms of the weight of gallic acid produced by the tannase treatment. Specifically, in the flavan-3-ol polymer, the content of the galloyl group is preferably 0.1 μg/mg or more, more preferably 1 μg/mg or more, still more preferably 10 μg/mg or more in terms of the weight of gallic acid produced by the tannase treatment. The content of the galloyl group may be 100 μg/mg or less, and 50 μg/mg or less in terms of the weight of gallic acid produced by the tannase treatment. In one aspect, in the flavan-3-ol polymer, the content of the galloyl group is preferably 0.1 to 100 μg/mg, more preferably 1 to 100 μg/mg, still more preferably 10 to 100 μg/mg, particularly preferably 10 to 50 μg/mg in terms of the weight of gallic acid produced by the tannase treatment.

The hydrolyzable tannin (A2) preferably has a galloyl group and/or an HHDP group. In addition to the galloyl group and/or the HHDP group, the hydrolyzable tannin (A2) may have a gallic acid residue other than these. Examples of the hydrolyzable tannin having a galloyl group and/or an HHDP group include gallotannin and ellagitannin. One of the hydrolyzable tannins may be used, and two or more thereof may be used in combination.

The hydrolyzable tannin is preferably a compound producing gallic acid and/or ellagic acid by tannase treatment or acid hydrolysis treatment. The hydrolyzable tannin producing gallic acid by the treatment typically has a galloyl group. The hydrolyzable tannin producing ellagic acid by the treatment typically has a gallic acid oligomer residue such as an HHDP group.

The production of gallic acid and/or ellagic acid can be confirmed by measuring the amounts of gallic acid and/or ellagic acid before and after the treatment for the compound. The conditions under which the hydrolyzable tannin is subjected to the tannase treatment or the acid hydrolysis treatment are not limited, and for example, the same conditions as those in the flavan-3-ol polymer can be adopted.

The gallotannin refers to a compound in which one or more gallic acids are bound to glucose. More specifically, the gallotannin is a compound in which a carboxyl group of gallic acid is esterified to one or more hydroxyl groups of glucose. The gallotannin has one or more galloyl groups, and produces gallic acid by tannase treatment or acid hydrolysis treatment.

In one aspect, the gallotannin is preferably a compound in which 3 to 5 molecules of gallic acid are bound to one molecule of glucose (preferably, gallotannin having 3 to 5 galloyl groups in one molecule, in which gallic acid is bound to 3 to 5 hydroxyl groups of glucose) since the compound has a high intestinal barrier function improvement effect, more preferably a compound in which four molecules of gallic acid are bound to one molecule of glucose (preferably, gallotannin having four galloyl groups, in which gallic acid is bound to four hydroxyl groups of glucose). The gallotannin is preferably one or more compounds selected from the group consisting of 1,4,6-tri-0-galloyl-β-D-glucose, 1,2,4,6-tetra-O-galloyl-β-D-glucose, 1,2,3,6-tetra-O-galloyl-β-D-glucose, 2,3,4,6-tetra-O-galloyl-β-D-glucose, and 1,2,3,4,6-penta-O-galloyl-β-D-glucose. Among these, 1,2,4,6-tetra-O-galloyl-β-D-glucose, 1,2,3,6-tetra-O-galloyl-β-D-glucose, and 2,3,4,6-tetra-O-galloyl-β-D-glucose are more preferable.

The ellagitannin is a hydrolyzable tannin producing ellagic acid by the tannase treatment or the acid hydrolysis treatment.

In view of the intestinal barrier function improvement effect, the ellagitannin preferably has a galloyl group and/or an HHDP group, more preferably an HHDP group, or a galloyl group and an HHDP group. When the ellagitannin has HHDP groups, the number of the HHDP groups per molecule is preferably 1 or 2, more preferably 2.

In terms of the intestinal barrier function improvement effect, the ellagitannin is preferably one or more compounds selected from the group consisting of tellimagrandin I, pedunculagin, praecoxin A, geraniin, stenophyllanin A, stenophyllanin B, casuarinin, and eugeniflorin D2, more preferably geraniin, stenophyllanin A, stenophyllanin B, and casuarinin.

The hydrolyzable tannin can be obtained, for example, by extracting a raw material containing a hydrolyzable tannin with water or hydrous ethanol, filtering the extract, removing the alcohol, and thereafter performing column purification.

A plant containing a hydrolyzable tannin can be used as the raw material of the hydrolyzable tannin. Examples of the plant containing a hydrolyzable tannin include fagaceae, lythraceae, myrtaceae, and rosaceae plants. These plants are rich in hydrolyzable tannins. The myrtaceae plant is preferably a plant such as a syzygium, eucalyptus, or kunzea plant. For example, an extract of eucalyptus leaves is rich in hydrolyzable tannins such as tellimagrandin I and gallotannin.

Each of the catechin gallate, epicatechin gallate, and gallocatechin gallate (A3) is a compound having a galloyl group. The compound (A3) can be obtained by, for example, purifying an extract from green tea leaves. Commercially available products can also be used as the compound (A3).

In the present invention, the “intestinal barrier function” means the function of protecting the intrusion (permeation) of foreign materials (for example, toxins such as endotoxin, inflammatory substances, and undigested products) into the body from the outside of intestinal epithelial cells (the inside of the intestine). The large intestine and the small intestine are contained in the intestine. A state where the intrusion of the foreign materials into the body from the outside of the intestinal epithelial cells is promoted as compared with the normal state is referred to as a state where the permeability of the foreign materials in the intestinal epithelial cells increases. The “intestinal barrier function improvement” means both suppression of the increase (rise) in the permeability of the foreign materials in the intestinal epithelial cells and decrease in the permeability of the foreign materials in the intestinal epithelial cells. In the present invention, the “intestinal barrier function improvement” is used to mean to include suppression of the decrease in the intestinal barrier function and enhancement of the decreased intestinal barrier function. For example, the intestinal barrier function is improved by normalizing or strengthening a tight junction bonding the intestinal epithelial cells together. In one aspect, the composition for improving intestinal barrier function according to the present invention may be used in order to improve the intestinal barrier function by normalizing or strengthening the tight junction in the intestinal epithelial cells.

The intestinal barrier function improvement effect is indicated by, for example, increase in the electrical resistance value (transepithelial electric resistance: TEER) of the intestinal epithelial cells or suppression of the decrease in the TEER. A substance increasing the TEER or suppressing the decrease thereof has the action of normalizing or strengthening the tight junction in the intestinal epithelial cells. The intestinal barrier function improvement effect is also indicated by decrease in the amount of a substance permeating from the intestinal side of the intestinal epithelial cells to the inside of the body.

The person skilled in the art can select a specific method of evaluating the intestinal barrier function improvement effect depending on the purpose. For example, as shown in Examples to be described later, a method for measuring TEER using an intestinal permeation model using human intestinal epithelial cells (Caco-2 cells) can be used. Specifically, inflammatory cytokine (TNFα, IL-1β, IFNγ and the like) is added into Caco-2 monolayer cultured cells to produce a state where the intestinal barrier function can be collapsed in human. If the addition of a test substance suppresses the decrease in the TEER as compared with the case where the substance is not added, the test substance can be evaluated to have the intestinal barrier function improvement effect.

As shown in Examples, the compounds having a gallic acid residue (A1) to (A3) are shown to suppress the decrease in the TEER due to the addition of inflammatory cytokine in an intestinal permeation model using Caco-2, to have an excellent intestinal barrier function improvement effect. As shown in Examples, the compound having a gallic acid residue exhibits a superior intestinal barrier function improvement effect to that of a compound having no gallic acid residue. For example, the compound (A3) exhibits a superior intestinal barrier function improvement effect to that of a compound having no galloyl group (catechin, epicatechin, or gallocatechin). The compounds having a gallic acid residue (A1) to (A3) can normalize or strengthen the tight junction in the intestinal epithelial cells to improve the intestinal barrier function.

As shown in Examples, the flavan-3-ol polymer having a galloyl group had the action of preventing or ameliorating a symptom of large intestine hyperalgesia due to the intestinal barrier function improvement action. The prevention or amelioration effect of abdominal discomfort is expected by preventing or ameliorating the large intestine hyperalgesia. Therefore, by improving the intestinal barrier function, the compound having a gallic acid residue such as the flavan-3-ol polymer having a galloyl group is useful, for example, for preventing or relieving the abdominal discomfort.

The composition for improving intestinal barrier function according to the present invention contains the compound having a gallic acid residue as the active ingredient to exhibit an excellent intestinal barrier function improvement effect. For this reason, the composition for improving intestinal barrier function according to the present invention is useful for preventing or ameliorating conditions or diseases against which improvement of the intestinal barrier function is effective, for example, conditions or diseases related to the intestinal barrier dysfunction. The intestinal barrier dysfunction includes deterioration of the intestinal barrier function. Examples of the conditions or diseases related to the intestinal barrier dysfunction include conditions or diseases caused by the intestinal barrier dysfunction, or conditions or diseases involving the intestinal barrier dysfunction. Examples of the conditions or diseases related to the intestinal barrier dysfunction include inflammatory bowel diseases, irritable bowel syndromes, systemic autoimmune diseases (rheumatoid arthritis, erythematosus and the like), allergies (food allergy, pollinosis and the like), and metabolic syndromes (obesity, type I or type II diabetes mellitus, hypertension, hyperlipidemia, non-alcoholic fatty liver diseases (NAFLD), arteriosclerosis and the like) (for example, Camilleri et al., Am, J Physiol Gastrointest Liver Physiol 303: G775-G785, 2012; Mu et al., Front. Immunol., Vol. 8, Article 598, 2017; Bischoff et, al., BMC Gastroenterology 2014 14:189).

More specific examples of symptoms of the conditions or diseases related to the intestinal barrier dysfunction include symptoms such as diarrhea, constipation, and discomfort (bloating, borborygmi, abdominal pain and the like) of the gut (abdominal part). The composition for improving intestinal barrier function according to the present invention has the action of improving the intestinal condition by improving the intestinal barrier function. Therefore, the composition for improving intestinal barrier function according to the present invention can control the intestinal function by the intestinal barrier function improvement, and is useful for preventing or ameliorating the symptom of the intestine as described above. In one aspect, the composition for improving intestinal barrier function according to the present invention may be used for intestinal regulation (in order to prevent or ameliorate, for example, diarrhea, constipation, and abdominal discomfort) by improving the intestinal barrier function. Among these, the composition for improving intestinal barrier function is preferably used in order to prevent or relieve the abdominal discomfort, and is useful also for preventing or relieving stress-induced abdominal discomfort. The intestinal barrier dysfunction is also associated with metabolic syndromes and the like (for example, the Bischoff et al., BMC Gastroenterology 2014 14:189 as described above). It is effective also in prevention or amelioration of the metabolic syndromes to improve the intestinal barrier function. Examples of the symptoms of the metabolic syndromes include abnormal glucose metabolism, abnormal lipid metabolism, increase in body fat, increase in visceral fat, increase in abdominal circumference fat, and higher blood pressure. Therefore, the composition for improving intestinal barrier function according to the present invention can improve the intestinal barrier function to contribute to improvement of glucose metabolism, improvement of lipid metabolism, decrease or suppression of increase in fat such as body fat, visceral fat, or abdominal circumference fat, amelioration of higher blood pressure and the like.

Herein, the “prevention of conditions or diseases” refers to enhancement of the resistance of a subject to the conditions or diseases, or delay or prevention of the onset of the conditions or diseases. Herein, the “amelioration of conditions or diseases” refers to recovery of a subject from the conditions or diseases, alleviation of the symptoms of the conditions or diseases, or delay or prevention of the progress of the conditions or diseases.

The composition of the present invention is applicable for both therapeutic use (medical use) and non-therapeutic use (non-medical use).

The composition for improving intestinal barrier function according to the present invention can be provided, for example, as a food or beverage, a pharmaceutical product, a quasi-pharmaceutical product, feed or the like, but it is not limited thereto. The composition for improving intestinal barrier function according to the present invention may be a food or beverage, a pharmaceutical product, a quasi-pharmaceutical product, or feed by itself, and may be a formulation or material such as an additive used therefor. The composition for improving intestinal barrier function according to the present invention may be provided as an agent as one example, but it is not limited thereto. The agent may be provided as a composition by itself or as a composition containing the agent.

In one aspect, the composition for improving intestinal barrier function according to the present invention is preferably an oral composition. The present invention can provide an oral composition having an excellent intestinal barrier function improvement action. Examples of the oral composition include a food or beverage, a pharmaceutical product, and a quasi-pharmaceutical product, and the oral composition is preferably a food or beverage.

The composition for improving intestinal barrier function according to the present invention may contain one or two or more ingredients (other ingredients) other than the above-described compound having a gallic acid residue as long as the effect of the present invention is not impaired.

In one aspect, for example, ingredients such as lactic acid bacteria, bifidobacteria, dietary fibers, and polysaccharides may be contained as the other ingredients. It is preferable that the lactic acid bacteria and the bifidobacteria can be orally ingested.

The dietary fiber may be any of a water-insoluble dietary fiber and a water-soluble dietary fiber. Examples of the water-insoluble dietary fiber include cellulose, lignin, hemicellulose, wheat bran, an apple fiber, a sweet potato fiber, and chitin. The water-soluble dietary fiber is roughly divided into a high viscous dietary fiber and a low viscous dietary fiber, and examples of the high viscous dietary fiber include pectin, konjac mannan, alginic acid, sodium alginate, guar gum, and agar. Among the dietary fibers generally known in Japan, the low viscous water-soluble dietary fiber is referred to as a dietary fiber material containing 50% by weight or more of a dietary fiber and dissolved in ordinary temperature water to provide a low viscous solution, i.e., an about 5% by weight aqueous solution having a viscosity of 20 mPa·s or less. Examples of the low viscous water-soluble dietary fiber include hardly digestible dextrin, polydextrose, partially hydrolyzed guar gum, and Litesse (polydextrose). Another examples of the low viscous water-soluble dietary fiber include dietary fiber materials satisfying low viscous and water-soluble properties. One of the dietary fibers may be used, and two or more thereof may be used.

Examples of the polysaccharides include oligosaccharides such as galactooligosaccharides, xylooligosaccharides, mannooligosaccharides, agarooligosaccharides, fructooligosaccharides, isomaltooligosaccharides, and raffinose. One of the oligosaccharides may be used, and two or more thereof may be used.

The composition for improving intestinal barrier function according to the present invention may contain optional additives and optional ingredients other than the above. The additives and the ingredients may be selected depending on the form of the composition for improving intestinal barrier function, and the like. The additives and the ingredients generally usable for a food or beverage, a pharmaceutical product, a quasi-pharmaceutical product, feed or the like can be used. Examples thereof include various additives which are acceptable in a food or beverage or pharmaceutically acceptable as an oral administration agent, such as an excipient, a lubricant, a stabilizer, a dispersant, a binder, a diluent, a flavour, a sweetener, a flavoring agent, and a colorant. For example, when the composition for improving intestinal barrier function according to the present invention is used as the oral composition, the oral composition can appropriately contain ingredients capable of being orally ingested other than the above as long as the effect of the present invention is not impaired. Such ingredients include vitamin, a vitamin-like substance, protein, amino acid, fat and oil, organic acid, a carbohydrate, a plant-derived raw material, an animal-derived raw material, a microorganism, an additive for food or beverage, and an additive for pharmaceutical product.

In addition to the above, ingredients such as materials used for a food or beverage, a pharmaceutical product, a quasi-pharmaceutical product, feed or the like can be appropriately blended depending on the use.

The form of the composition for improving intestinal barrier function according to the present invention is not limited as long as the effect of the present invention is obtained. Examples of the form include tablets, pills, granules, fine granules, chews, capsules (including soft and hard capsules), liquids, chewable tablets, and beverages. The form may be other food form. These dosage forms may be prepared by using conventional methods commonly known in the art.

In one aspect, when the composition for improving intestinal barrier function according to the present invention is used as a food or beverage, ingredients (for example, a material for food or beverage, and an additive used as necessary) usable for the food or beverage can be blended with the compound having a gallic acid residue to provide various foods or beverages (compositions for food or beverage). The food or beverage is not limited. Examples thereof include general foods and beverages, health foods, foods with function claims, foods for specified health uses, foods for the sick, food additives, and raw materials thereof. The form of the food or beverage is not also limited, and examples thereof include various formulation forms such as solid oral formulations (such as tablets, coating tablets, fine granules, granules, powders, pills, capsules (including soft and hard capsules), dry syrup agents, and chewable tablets); and liquid oral formulations (such as internal liquid formulations and syrups). In one aspect of the present invention, the food or beverage may contain one or two or more of the lactic acid bacteria, the bifidobacteria, the dietary fibers, and the polysaccharides.

When the composition for improving intestinal barrier function according to the present invention is used as the pharmaceutical product or the quasi-pharmaceutical product, an additive such as a pharmaceutically acceptable excipient can be blended with the compound having a gallic acid residue, to provide the pharmaceutical products (pharmaceutical compositions) or quasi-pharmaceutical products (quasi-pharmaceutical product compositions) of various dosage forms. The form of administration of the pharmaceutical product or quasi-pharmaceutical product is preferably oral administration. The dosage form of the pharmaceutical product or quasi-pharmaceutical product may be a dosage form suitable to the form of administration. Examples of the dosage form of the oral pharmaceutical product or quasi-pharmaceutical product include solid oral formulations such as tablets, coating tablets, fine granules, granules, powders, pills, capsules (including soft and hard capsules), dry syrup agents, and chewable tablets; and liquid oral formulations such as internal liquid formulations and syrups.

The tablets, the pills, and the granules may be in dosage forms conventionally coated as necessary such as sugar-coated tablets, gelatin-coated preparations, enteric-coated preparations, and film-coated agents. The tablets may be in the form of double or multiple layer tablets.

When the composition for improving intestinal barrier function according to the present invention is used as a food or beverage, a pharmaceutical product, a quasi-pharmaceutical product, feed or the like, the producing method is not limited, and the composition for improving intestinal barrier function can be produced by a general method using the compound having a gallic acid residue. The present invention also includes use of at least one compound selected from the group consisting of the (A1) to (A3) for the manufacture of the composition for improving intestinal barrier function.

The composition for improving intestinal barrier function according to the present invention may be labeled with one or more of the following information on a package, container, or package insert: usage, types of active ingredients, the above-described effects, and instructions for use (e.g., ingestion method or administration method). The composition for improving intestinal barrier function according to the present invention may be labeled as having an intestinal barrier function improvement action or an action based on the intestinal barrier function improvement action. The composition for improving intestinal barrier function may be labeled as having an intestinal regulation action, for example.

The intestinal regulation action may be an intestinal regulation action based on improvement of intestinal barrier function, and is not limited. Examples of the labeling as having an intestinal regulation action include “for one tending to be constipated or have diarrhea”, “for one worrying about gut function”, “for one readily feeling discomfort of gut”, “improvement of bowel movement”, “improvement of stool state”, “amelioration of defecation frequency”, “amelioration of defecation output”, “gut feels better”, “controlling gut function”, “controlling intestinal function”, “amelioration of discomfort in gut”, “reduction of generation of gas”, “reduction of abdominal bloatedness”, and “amerilation of borborygmi”. The composition for improving intestinal barrier function according to the present invention may include one or two or more of such labelings.

The content of the compounds having a gallic acid residue (A1) to (A3) in the composition for improving intestinal barrier function according to the present invention can be appropriately set depending on the form of the composition and the like. In one aspect, when the composition for improving intestinal barrier function is used as the oral compositions such as a food or beverage, a pharmaceutical product, and a quasi-pharmaceutical product, the total content of the compounds having a gallic acid residue (A1) to (A3) is preferably 0.0001% by weight or more, more preferably 0.01% by weight or more in the composition. The total content of the compounds having a gallic acid residue (A1) to (A3) is preferably 80.0% by weight or less, more preferably 20.0% by weight or less in the composition. In one aspect, the total content of the compounds (A1) to (A3) is preferably 0.0001 to 80.0% by weight, more preferably 0.01 to 20.0% by weight in the composition for improving intestinal barrier function. When the composition contains two or more compounds having a gallic acid residue (A1) to (A3), the total content refers to the total amount of these compounds.

The content of the compounds having a gallic acid residue (A1) to (A3) can be measured according to known methods, and for example, an HPLC method can be used.

The composition for improving intestinal barrier function according to the present invention can be ingested or administered by a suitable method depending on the form. It is preferable that the composition for improving intestinal barrier function according to the present invention is orally administered or orally ingested.

The amount of the composition for improving intestinal barrier function according to the present invention ingested (which may also be referred to as the amount administered) is not limited, and may be an amount such that the intestinal barrier function improvement effect is obtained. The amount ingested may be appropriately set according to the form of administration or administration method, for example. As one aspect, for the amount of the composition for improving intestinal barrier function ingested when the composition is orally administered to or allowed to be ingested by a human (adult) subject (for example, weight: 60 kg), the total amount of the compounds (A1) to (A3) ingested is preferably 0.01 to 5000 mg, more preferably 0.1 to 4000 mg, still more preferably 1 to 3000 mg per day. It is preferable that the amount is orally administered or allowed to be ingested, for example, once daily or in a divided manner two to three times daily. In one aspect, when the composition for improving intestinal barrier function is allowed to be ingested by the human (adult) subject for the purpose of obtaining the intestinal barrier function improvement effect, the composition for improving intestinal barrier function can be allowed to be orally ingested by or administered to the subject so that the total amount of the compounds (A1) to (A3) ingested is within the above range. In one aspect of the present invention, in the case of the human (adult) (for example, weight; 60 kg), for the amount of the composition for improving intestinal barrier function ingested, the amount of the compound (A1) (flavan-3-ol polymer having a galloyl group) ingested is preferably 1 to 2000 mg, more preferably 10 to 1500 mg, still more preferably 30 to 1000 mg, particularly preferably 100 to 1000 mg per day. When the amount of the compound (A1) ingested is within the above range, an excellent intestinal barrier function improvement effect can be obtained. The amount is preferably orally administered or allowed to be ingested, for example, once daily or in a divided manner two to three times daily. In one aspect, the composition for improving intestinal barrier function according to the present invention may be an oral composition for allowing the adult to ingest or administering the above amount of the compound (A1) per day per weight of 60 kg to the adult.

In one aspect, the composition for improving intestinal barrier function according to the present invention preferably contains the compound having a gallic acid residue in an amount such that the desired effect of the present invention is obtained, that is, an effective amount in consideration of the form of administration, administration method and the like thereof. As one aspect, for example, when the composition for improving intestinal barrier function is an oral composition such as a food or beverage or an oral pharmaceutical product, the total content of the compounds (A1) to (A3) in the amount of the composition ingested per adult (for example, weight: 60 kg) per day is preferably 0.01 to 5000 mg, more preferably 0.1 to 4000 mg, still more preferably 1 to 3000 mg. In one aspect, when the composition for improving intestinal barrier function is an oral composition, the content of the compound (A1) in the amount of the composition ingested per adult (for example, weight: 60 kg) per day is preferably 1 to 2000 mg, more preferably 10 to 1500 mg, still more preferably 30 to 1000 mg, particularly preferably 100 to 1000 mg.

A greater intestinal barrier function improvement effect can be expected by continuous ingestion (administration) of the compounds (A1) to (A3) which are active ingredients of the composition for improving intestinal barrier function according to the present invention. In a preferable aspect, the composition for improving intestinal barrier function according to the present invention is continuously ingested. In one embodiment of the present invention, it is preferable that the composition for improving intestinal barrier function is continuously ingested for a week or more.

A subject (hereinafter, merely referred to as an “administration subject”) to whom the composition for improving intestinal barrier function according to the present invention is administered or by whom the composition is allowed to be ingested is preferably human or non-human animal, more preferably mammal (human or non-human mammal), still more preferably human. The administration subject in the present invention is preferably a subject requiring or desiring the intestinal barrier function improvement. Suitable examples of the subject include a subject having a reduced intestinal barrier function and a subject desiring the prevention or amelioration of conditions or diseases related to intestinal barrier dysfunction.

The present invention also includes the following method for improving intestinal barrier function.

A method for improving intestinal barrier function, the method including: administering at least one compound having a gallic acid residue selected from the group consisting of the following (A1) to (A3) to a subject:

(A1) a flavan-3-ol polymer having a galloyl group;

(A2) a hydrolyzable tannin; and

(A3) at least one compound selected from the group consisting of catechin gallate, epicatechin gallate, and gallocatechin gallate.

Use of at least one compound having a gallic acid residue selected from the group consisting of the above (A1) to (A3) for improving intestinal barrier function.

The method and the use may be therapeutic or non-therapeutic. The “non-therapeutic” is a concept which does not include medical activities, i.e., a concept which does not include surgery, therapy or diagnosis.

The amount of the compound having a gallic acid residue administered may be an amount such that the intestinal barrier function improvement effect is obtained, i.e., an effective amount, and is not limited. For example, the above-described amount is preferably administered. An administration route is preferably oral administration. The compound having a gallic acid residue may be administered as it is, or a composition containing the above-described compound having a gallic acid residue may be administered. For example, the above-described composition for improving intestinal barrier function according to the present invention can be administered. The compound having a gallic acid residue, the subject (administration subject), the administration method, the amount administered, preferable aspects thereof and the like are the same as those in the above-described composition for improving intestinal barrier function. In one aspect, when the compound (A1) (flavan-3-ol polymer having a galloyl group) is orally administered to, for example, a human (adult) subject, the daily amount of the compound (A1) administered is preferably 1 to 2000 mg, more preferably 10 to 1500 mg, still more preferably 30 to 1000 mg, yet still more preferably 100 to 1000 mg per weight of 60 kg. In the above method and use, it is preferable that one or more compounds having a gallic acid residue (A1) to (A3) are continuously administered for a week or more.

### EXAMPLES

The following provides Examples which more specifically describe the present invention. The present invention is not limited to these Examples.

### Example 1

Purification of flavan-3-ol polymer having galloyl group (Hereinafter, flavan-3-ol polymer is also referred to as OPC.)

A commercially available grape seed extract containing 81% or more of oligomeric procyanidin (flavan-3-ol polymer) as a standard was dissolved in water, and the solution was subjected to liquid-liquid separation three times using ethyl acetate. The obtained two fractions were concentrated under reduced pressure, and freeze-dried to obtain a dry powder. A water layer containing OPC was fractionated by a method described in Literature (Biosci. Biotechnol. Biochem., 73, 1274-1279 (2009)), to obtain a grape-derived purified OPC fraction having a higher purity.

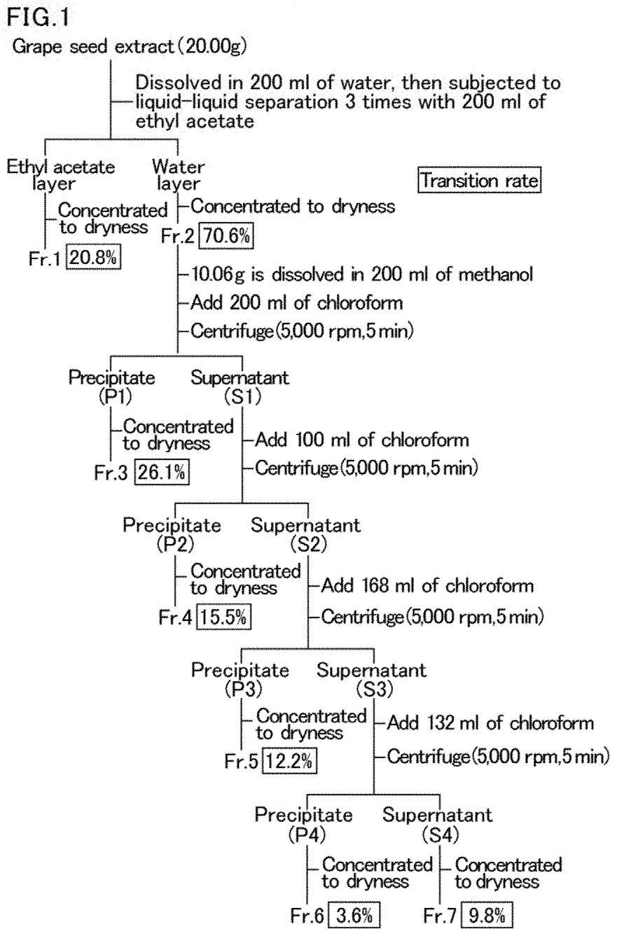

A specific procedure will be described later. FIG. 1 is a flow chart showing a procedure of purifying a flavan-3-ol polymer (OPC) having a galloyl group from a grape seed extract.

A transition rate (%) means “100×yield (g)/starting material (g).”

The grape seed extract (20.00 g) was dissolved in water (200 mL), and then subjected to liquid-liquid separation three times with ethyl acetate (200 mL). An ethyl-acetate layer was concentrated to dryness to obtain a fraction 1 (Fr.1) (transition rate: 20.8%). The water layer was concentrated to dryness to obtain a fraction 2 (Fr.2) (transition rate: 70.6%). The fraction 2 (10.06 g) was dissolved in methanol (200 mL), and chloroform (200 mL) was added thereto. The obtained solution was centrifuged (5000 rpm, 5 minutes) to be separated into a precipitate (P1) and a supernatant (S1). The precipitate (P1) was concentrated to dryness to obtain a fraction 3 (Fr.3) (transition rate: 26.1%).

Chloroform (100 mL) was added to the supernatant (S1), followed by centrifugal separation (5000 rpm, 5 minutes), to be separated into a precipitate (P2) and a supernatant (S2). The precipitate (P2) was concentrated to dryness to obtain a fraction 4 (Fr.4) (transition rate: 15.5%).

Chloroform (168 mL) was added to the supernatant (S2), followed by centrifugal separation (5000 rpm, 5 minutes), to be separated into a precipitate (P3) and a supernatant (53). The precipitate (P3) was concentrated to dryness to obtain a fraction 5 (Fr.5) (transition rate: 12.2%).

Chloroform (132 mL) was added to the supernatant (S3), followed by centrifugal separation (5000 rpm, 15 minutes), to be separated into a precipitate (P4) and a supernatant (S4).

The precipitate (P4) was concentrated to dryness to obtain a fraction 6 (Fr.6) (transition rate: 3.6%). The supernatant (S4) was concentrated to dryness, to obtain a fraction 7 (Fr.7) (transition rate: 9.8%).

The obtained fraction 5 (Fr.5) was taken as a grape-derived purified OPC fraction.

### Example 2

### Analysis of OPC

The purity of the grape-derived purified OPC fraction obtained in Example 1 was calculated according to the method described in Japanese Patent No. 4659407. The grape-derived purified OPC fraction was subjected to acid hydrolysis treatment according to Japanese Patent No. 4659407, and analyzed under the following analysis conditions.

(Analysis conditions of high performance liquid chromatography (HPLC))

### Detection: 520 nm

Column: YMC-Pack ODS-A (5 μm, 6.0 mm i.d.×150 mm, manufactured by YMC Co., Ltd.)
Solvent (mobile phase): acetic acid:methanol:distilled water=15:17.5:67.5
Column temperature: 40° C.
Flow rate: 1 mL/min
Analysis time: 12 min
Injection volume: 5 μL

Procyanidin B1 (AdooQ BioScience, purity: 99% or more) was used as a standard substance of OPC.

### (Purity Calculating Formula of OPC)

OPC purity (%)−100×(concentration of cyanidin derived from grape-derived purified OPC fraction subjected to acid hydrolysis treatment)/(concentration of cyanidin derived from procyanidin B1subjected to acid hydrolysis treatment)

When the purity of OPC in the grape-derived purified OPC fraction prepared in Example 1 was calculated by using the formula, the purity was 91%.

### Example 3

### Tannase Treatment of Grape-Derived Purified OPC

20 mg of the grape-derived purified OPC fraction produced in Example 1 and 20 mg of tannase (manufactured by Wako Pure Chemical Industries, Ltd., derived from Aspergillus oryzae) were dissolved in a citrate buffer solution (pH: 5.5) so that the final concentrations thereof were set to 1 mg/mL. The solution was reacted at 30° C. overnight (for about 16 hours) for tannase treatment. A part of the obtained reaction solution was used as the sample after the tannase treatment for measurement of gallic acid to be described later. The reaction solution was applied to Sep-Pak Vac 20 cc (5 g) C18 Cartridges (manufactured by Waters Corporation). After washing with 400 mL of distilled water in order to remove a highly-polar ingredient, a citric salt, tannase, and gallic acid, OPC was eluted with 60 mL of 100% methanol. The obtained 100% methanol eluate was concentrated under reduced pressure, and freeze-dried, to obtain a dry powder. The grape-derived purified OPC subjected to the tannase treatment was taken as grape-derived purified OPC (having no galloyl group). The same steps as the above were performed except that tannase was not added, to prepare a powder, which was taken as grape-derived purified OPC (having a galloyl group).

Measurement of Gallic Acid Produced by Tannase Treatment

The grape-derived purified OPC fraction produced in Example 1 was dissolved in a citrate buffer solution (pH: 5.5) so that the concentration was set to 1 mg/mL to obtain a sample before tannase treatment.

The sample before the tannase treatment and the sample after the tannase treatment obtained above were applied to a 0.45-μm filter, and the amount of gallic acid was then analyzed by HPLC. The amount of gallic acid was determined with the external standard method.

HPLC analysis conditions for analysis of gallic acid Detection wavelength: UV (280 nm)
Column: Capcell pak C18 (ϕ4.6×150.0 mm, manufactured by Shiseido Company, Limited)
Solvent A: 0.05% trifluoroacetic acid-containing aqueous solution
Solvent B: 0.05% trifluoroacetic acid-containing 90% acetonitrile aqueous solution

### Gradient:

Solvent B concentration (volume) %: 10%, 0-5 min; 10-100%, 5-20 min; 100%, 20-25 min; 10%, 25.01-30 min
Column temperature: 40° C.
Flow rate: 1.0 mL/min

### Injection: 10 μL

The amount of gallic acid produced by the tannase treatment was obtained according to the following calculating formula from the amounts of gallic acid in the sample after the tannase treatment and the sample before the tannase treatment.

Amount of gallic acid produced by tannase treatment=(Amount of gallic acid after treatment)−(Amount of gallic acid before treatment)

In the formula, the amount of gallic acid after the treatment is the amount of gallic acid in the sample after the tannase treatment, and the amount of gallic acid before the treatment is the amount of gallic acid in the sample before the tannase treatment.

In the grape-derived purified OPC fraction, gallic acid was not detected in the sample before the tannase treatment, and 45 μg/mg of gallic acid was detected in the sample after the tannase treatment. This gallic acid (45 μg/mg) is gallic acid produced by the tannase treatment from the grape-derived purified OPC (having a galloyl group) (45 μg of gallic acid is produced per mg of the grape-derived purified OPC (having a galloyl group)). Therefore, the grape-derived purified OPC (having a galloyl group) had 45 μg/mg of a galloyl group in terms of the weight of gallic acid produced by the tannase treatment. In the grape-derived purified OPC (having no galloyl group), the galloyl group is removed in an amount corresponding to the above amount.

### Example 4

Comparative evaluation of intestinal barrier function improvement action depending on the presence or absence of a galloyl group was performed.

### (Method for Evaluating Intestinal Barrier Function Improvement Action)

Caco-2 cells were cultured for three weeks at 37° C. in Transwell (manufactured by Millicell Corporation) using DMEM (Dulbecco's modified Eagle's medium). A medium was removed from a plate of cultured Caco-2 cells, and the well was washed 3 times with serum-free DMEM. The well was filled with the medium. Then, the transepithelial electrical resistance (TEER) of Caco-2 monolayer cells was measured by Millicell-ERS (manufactured by Millipore Corporation). Cells (TEER≥1000 Ω·cm²) determined that a sufficient tight junction was formed were selected, and used for the next screening. Then, to test solutions (media) on both apical and basolateral membrane sides, a sample, TNFα (40 ng/mL), IL-1β (20 ng/mL), and IFNγ (10 ng/mL) were added, followed by cultivating for 48 hours. The sample was dissolved in dimethyl sulfoxide (DMSO), and the solution was then added to the test solutions. In this case, a well to which inflammatory cytokine (TNFα, IL-1β, and IFNγ) and the sample were not added was provided as normal. A well to which inflammatory cytokine was added and the sample was not added was provided as control. After the cultivation, TEER was measured again, to evaluate whether the sample suppressed the reduction (decrease) of TEER due to the inflammatory cytokine.

The TEER reduction suppression ratio (%) due to the sample was determined according to the following formula from the TEER values of the well to which the sample was added, normal, and control.

### (Calculating Formula of TEER Reduction Suppression Ratio)

TEER reduction suppression ratio (%)−100×((TEER of well to which sample is added)−(TEER of control))/((TEER of normal)−(TEER of control))

In this evaluation system, as the TEER reduction suppression ratio (%) is higher, an intestinal barrier function improvement effect is higher.

The evaluated samples are compounds shown in the following Table 1. Catechin gallate, epicatechin gallate, and gallocatechin gallate were used as gallate type compounds of catechin, epicatechin, and gallocatechin, respectively (all manufactured by Wako Pure Chemical Industries, Ltd.). The grape-derived purified OPC (having a galloyl group) and the grape-derived purified OPC (having no galloyl group) prepared in Example 3 were used.

TABLE 1

| Evaluation | |
|---|---|
| No. | Sample |
| 1 | Catechin |
| | Catechin gallate |
| 2 | Epicatechin |
| | Epicatechin gallate |
| 3 | Gallocatechin |
| | Gallocatechin gallate |
| 4 | Grape-derived purified OPC (having no |
| | galloyl group) |
| | Grape-derived purified OPC (having a |
| | galloyl group) |

In Evaluation Nos. 1 to 3, each sample was added so that a sample concentration in the test solution was set to 10 μmol/L (10 μM).

Each sample used for evaluation suppressed the reduction of TEER due to the inflammatory cytokine. As a result of the evaluation, in the catechin, the epicatechin, and the gallocatechin, the gallate type compound was found to have a higher intestinal barrier improvement function than that of a non-gallate type compound. The results are shown in Table 2.

TABLE 2

| | | | TEER | |
|---|---|---|---|---|
| | | | reduction | |
| Evaluation | | Concentration | suppression | Relative |
| No. | Sample | (μM) | ratio (%) | value |
| 1 | Catechin | 10 | 15 | 2.7 |
| | Catechin | 10 | 40 | |
| | gallate | | | |
| 2 | Epicatechin | 10 | 14 | 2.5 |
| | Epicatechin | 10 | 35 | |
| | gallate | | | |
| 3 | Gallocatechin | 10 | 19 | 3.2 |
| | Gallocatechin | 10 | 60 | |
| | gallate | | | |

Relative values in Table 2 show the relative value of the TEER reduction suppression ratio (%) of the gallate type compound when the TEER reduction suppression ratio (%) of the non-gallate type compound is taken as 1 in each evaluation. For example, in Evaluation 1, the relative value of catechin gallate (gallate type compound) is a relative value of the TEER reduction suppression ratio of catechin gallate when the TEER reduction suppression ratio of catechin (non-gallate type compound) is taken as 1.

In Evaluation No. 4, the test was performed at a sample (grape-derived purified OPC) concentration of 0.01 μg/mL, 0.1 μg/mL, 1 μg/mL, or 10 μg/mL in the test solution. Sample concentrations (IC₅₀) suppressing the reduction of TEER due to the inflammatory cytokine to 50% were calculated for the grape-derived purified OPC. The sample concentrations (IC₅₀) are shown in Table 3.

When the concentration of the grape-derived purified OPC in the test solution was set to, for example, 1 μg/mL, the TEER reduction suppression ratio of the grape-derived purified OPC (having a galloyl group) was 65%, and the TEER reduction suppression ratio of the grape-derived purified OPC (having no galloyl group) was 51%.

TABLE 3

| Evaluation | | IC₅₀ |
|---|---|---|
| No. | Sample | (μg/mL) |
| 4 | Grape-derived purified OPC (having no | 0.9084 |
| | galloyl group) | |
| | Grape-derived purified OPC (having a | 0.0721 |
| | galloyl group) | |

### Example 5

### Evaluation of Intestinal Barrier Function Improvement Action of Hydrolyzable Tannin

Intestinal barrier function improvement action was evaluated using a hydrolyzable tannin having a gallic acid residue as a sample. Hydrolyzable tannins purified from plants were used (purity: 90% or more in any of the hydrolyzable tannins). For example, tellimagrandin I and gallotannin were purified from Eucalyptus cypellocarpa leaves. Geraniin was purified from Geanium thunbergii leaves. Other hydrolyzable tannins purified from myrtaceae plants such as Kunzea ambigua leaves and Melaleuca alternifolia were used. The hydrolyzable tannins were purified from the plants by the method described in p. 76 to 95 of Literature (KASAJIMA Naoki, “Ingredient Research of Kunzea Ambigua and Eucalyptus cypellocarpa” (publication: September, 2005, doctoral dissertation, Okayama University)).

The intestinal barrier function improvement action was evaluated by the same method (the method for evaluating intestinal barrier function improvement action described in Example 4) as that in Example 4. In Example 5, the sample was added into the test solution so that a sample concentration was set to 10 μmol/L. The TEER reduction suppression ratio (%) of the sample was obtained according to the formula from the TEER values of a well to which the sample was added, normal, and control in the same manner as in Example 4.

The names, evaluation concentrations (sample concentrations in the test solution), and evaluation results (TEER reduction suppression ratios) of the evaluated compounds (samples) are shown in Table 4. For the gallic acid residue contained in the compound, the number of galloyl groups and the number of hexahydroxydiphenoyl groups (HHDP groups) in one molecule of the compound are shown in Table 4.

For comparison, in place of the sample, quercetin (Funakoshi Co., Ltd.) was added at 10 μmol/L into the test solution, to evaluate intestinal barrier function improvement action according to the above method. The TEER reduction suppression ratio of quercetin (10 μmol/L) was 38%. For the evaluated compound, intestinal barrier function improvement action superior to that of quercetin was found at the same concentration as that of quercetin.

TABLE 4

| | | Number of | Number of | | TEER |
|---|---|---|---|---|---|
| | | galloyl | HHDP | Concen- | reduction |
| | | groups/ | groups/ | tration | suppression |
| | Compound | molecule | molecule | (μM) | ratio (%) |
| Gallo- | 1,4,6-tri- | 3 | 0 | 10 | 56 |
| tannin | O-galloyl- | | | | |
| | β-D- | | | | |
| | glucose | | | | |
| | 1,2,3,6- | 4 | 0 | 10 | 98 |
| | tetra-O- | | | | |
| | galloyl- | | | | |
| | β-D- | | | | |
| | glucose | | | | |
| | 2,3,4,6- | 4 | 0 | 10 | 93 |
| | tetra-O- | | | | |
| | galloyl- | | | | |
| | β-D- | | | | |
| | glucose | | | | |
| | 1,2,4,6- | 4 | 0 | 10 | 80 |
| | tetra-O- | | | | |
| | galloyl- | | | | |
| | β-D- | | | | |
| | glucose | | | | |
| | 1,2,3,4,6- | 5 | 0 | 10 | 64 |
| | penta-O- | | | | |
| | galloyl- | | | | |
| | β-D- | | | | |
| | glucose | | | | |
| Ellagi- | Tellima- | 2 | 1 | 10 | 65 |
| tannin | grandin I | | | | |
| | Pedunculagin | 0 | 2 | 10 | 72 |
| | Praecoxin A | 0 | 1 | 10 | 74 |
| | Geranlin | 1 | 2 | 10 | 83 |
| | Steno- | 1 | 2 | 10 | 96 |
| | phyllanin A | | | | |
| | Steno- | 1 | 2 | 10 | 126 |
| | phyllanin B | | | | |
| | Casuarinin | 1 | 2 | 10 | 82 |

The structures of the evaluated compounds are shown below.

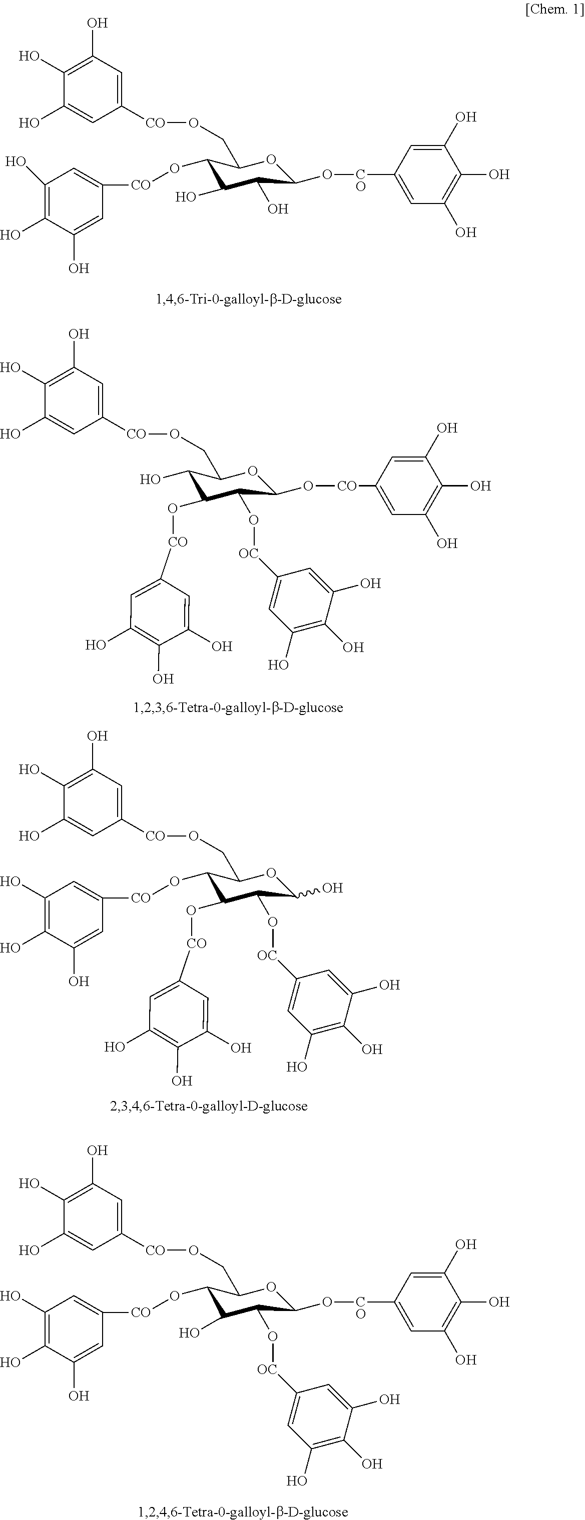

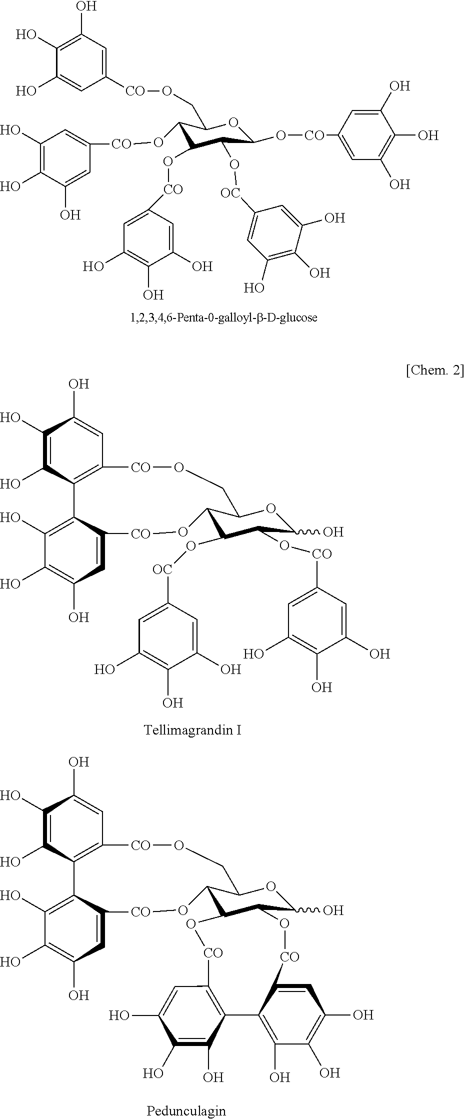

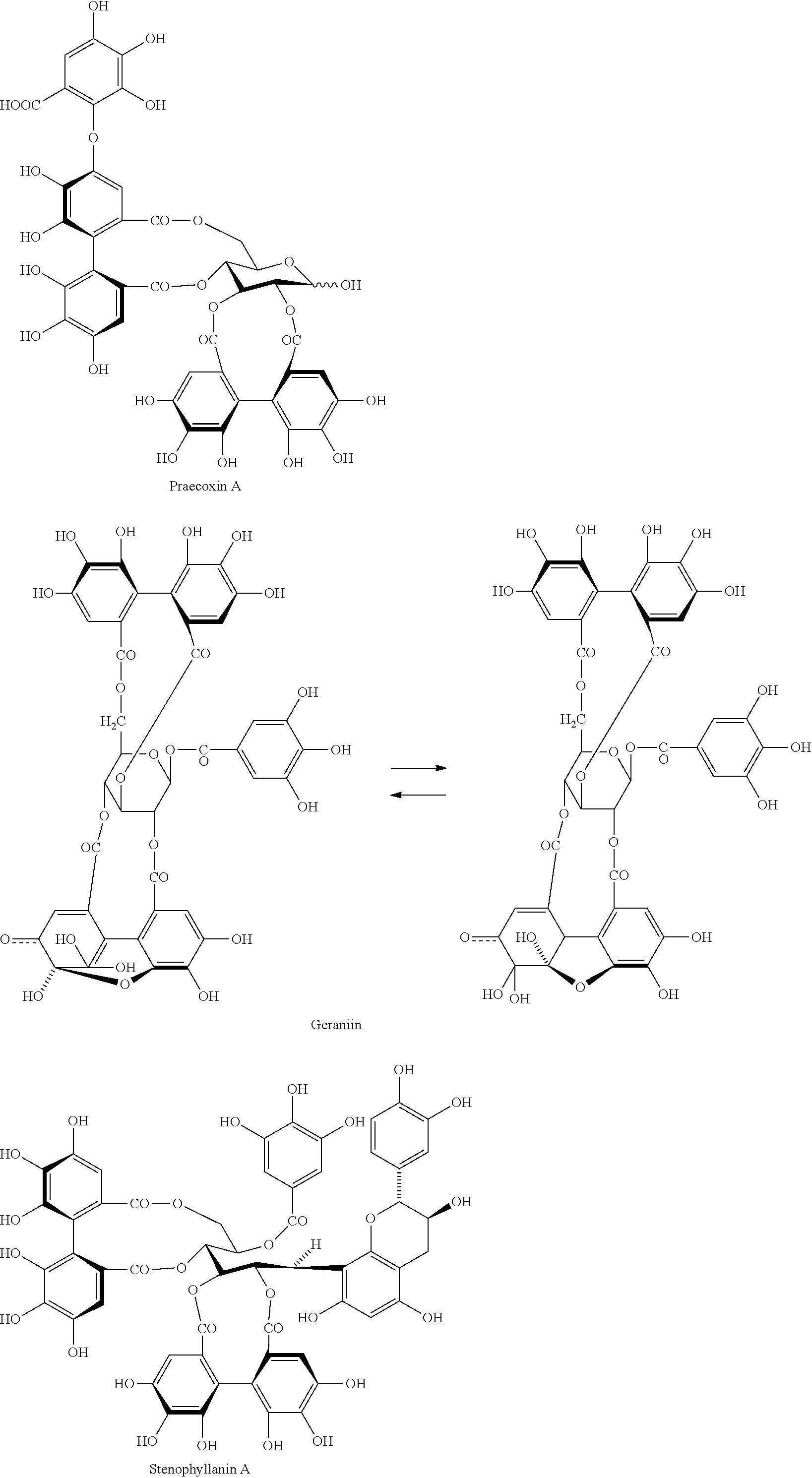

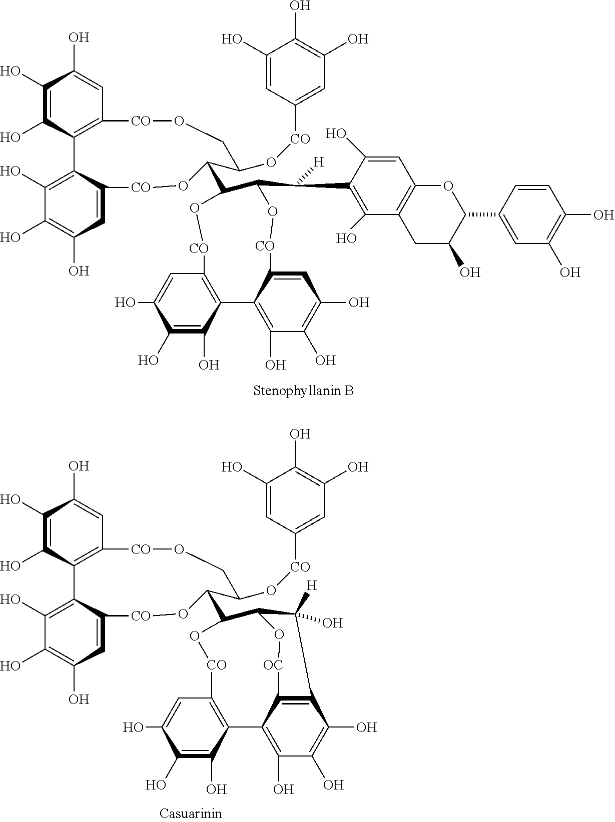

### Example 6

Effect of grape seed-derived flavart-3-ol polymer (OPC) having galloyl group on intestinal permeability and stress-related large intestine hyperalgesia A series of animal experiments was performed based on a plan approved by the relevant chief through evaluation of the in-house animal experiment committee, in compliance with the animal welfare management laws and other related laws and regulations.

### (Method)

A commercially available grape seed extract containing 83% of OPC was used for administering a flavan-3-ol polymer (OPC) having a galloyl group. When the OPC contained in the grape seed extract was subjected to 1.0 tannase treatment by the method described in Example 3, gallic acid was produced, so that the OPC having a galloyl group was confirmed. 25.5 μg of gallic acid was produced per mg of the OPC.

Male Sprague-Dawley rats weighing about 300 g were divided into three groups.

Group 1: no stress+vehicle (N=7), Group 2: stress+vehicle (N=6), Group 3: stress+grape seed extract (N=6)

The grape seed extract was administered to the rats of group 3, and vehicle (distilled water) was administered to the rats of groups 1 and 2.

The vehicle or the grape seed extract was orally administered to the rats for one week (once daily) before the rats were subjected to stress. A solution of 10 mg/mL in which the grape seed extract was dissolved in distilled water was administered. The amount of the grape seed extract administered was set to 83 mg/kg (10 mL/kg) per day per weight in terms of OPC having a galloyl group. 10 mL/kg of distilled water was administered per weight to the rats of groups 1 and 2. During the test period, the rats were fed with free access to feed (CRF-1, Oriental Yeast Co., ltd.) and water.

The vehicle or the grape seed extract was administered to the rats for one week as described above, and the rats of groups 2 and 3 were then subjected to a water avoidance stress (WAS) load.

The rats were placed on a platform located at the center of a water tank for 1 hour for a stress treatment for three days (once daily) to induce large intestine hyperalgesia. During the stress treatment period (three days), the vehicle or the grape seed extract was administered 1.5 hours before the rats were subjected to stress.

The large intestine hyperalgesia was evaluated by the following method. Under anesthesia, a balloon catheter (silicone catheter, 2.0 mm, Terumo Corporation) was transanally inserted 2 cm into the rat, and an electrode (Teflon (registered trademark)-coated stainless steel, 0.05 mm, MT Research Institute, Inc.) was inserted 2 mm into left side exterior oblique muscles. Then, the rat was awakened in a state where it was placed in a ballman cage. To induce large intestine pain, the balloon catheter was gradually enlarged by pouring water after 30 minutes of treatment. Abdominal muscle contraction induced by large intestine pain was observed by using an electromyogram, and a pain threshold was measured. The pain threshold was measured twice before and after stress sessions. The pre-stress measurement was performed immediately before the stress treatment (before stress) on the first day. In the measurement after stress, a balloon catheter and an electrode were inserted into the rats as described above 24 hours after the last stress treatment (the third day of the stress treatment), and a pain threshold was measured after 30 minutes. The vehicle or the grape seed extract was orally administered 1.5 hours before the pain threshold was measured after stress.

In the evaluation results of the pain threshold, the pain threshold before stress was taken as 100%, and change in the pain threshold after stress (100×(pain threshold after stress)/(pain threshold before stress)) (%) was calculated.

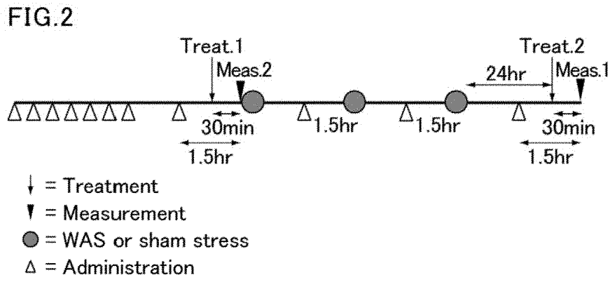

A test schedule of Example 6 is shown in FIG. 2. In FIG. 2, an arrow (↓) represents surgery in which a balloon catheter is inserted into a rat; an inverse triangle (▾) represents measurement of a pain threshold; a circle (●) represents WAS (water avoidance stress) or no stress (sham stress); and a triangle (Δ) represents administration of the grape seed extract or vehicle. Treat.1 and Meas.1 represent the insertion surgery of the balloon catheter and the measurement of the pain threshold before stress, respectively. Treat.2 and Meas.2 represent the insertion surgery of the balloon catheter and the measurement of the pain threshold after stress, respectively.

After the evaluation of the large intestine hyperalgesia, the intestinal permeability of the large intestine and the expression level of a tight junction protein were measured by the following method.

The large intestine hyperalgesia after stress was evaluated, and the contents of the large intestine were then washed under anesthesia. The upper part of the large intestine was ligated at two points, to produce a 4 cm loop. 1 mL of a 1.5% Evans blue solution was injected thereto, and allowed to stand for 15 minutes. The ligation site was taken out, and washed with PBS and N-acetyl-cysteine. Evans blue permeating was extracted with 2 mL of N,N-dimethylformamide. Then, the absorbance was measured to determine the amount of Evans blue permeating. The intestinal permeability (mg/g tissue) of the large intestine was calculated by correcting the amount (mg) of Evans blue permeating by the weight (g) of the ligation site of the large intestine.

The expression level of tight junction protein Claudin-2 was analyzed by the Wes system of ProteinSimple. A 1-cm tissue was extracted from under the ligation site of the large intestine, and a sample was prepared using a tissue dissolving solution (1% SDS, 1% Triton, 1% sodium deoxycholate in PBS).

### (Results)

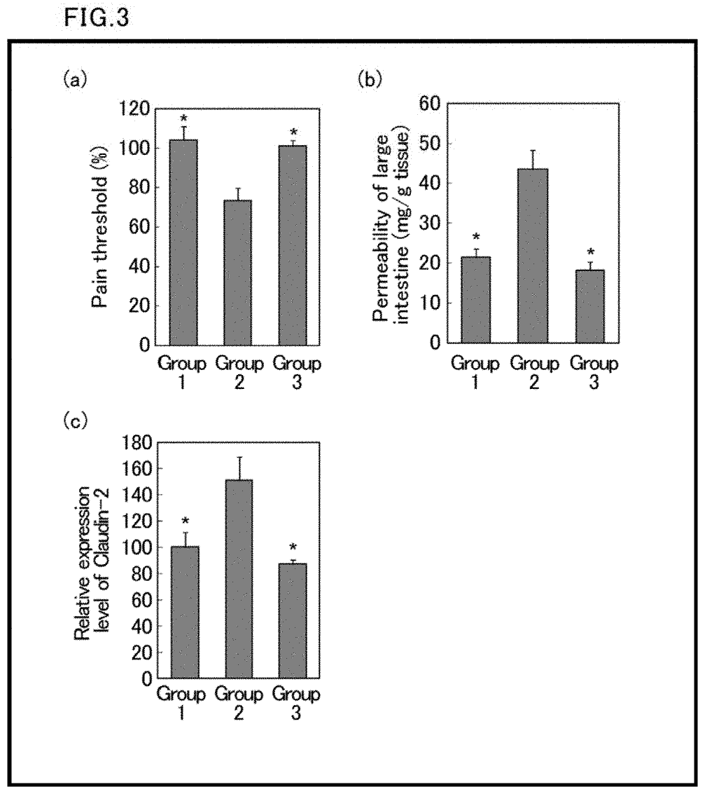

FIGS. 3(a) to 3(c) show the results of examining the effect of the flavan-3-ol polymer having a galloyl group on the pain threshold of the large intestine, the intestinal permeability of the large intestine, and the expression of Claudin-2, respectively. FIG. 3(a) shows the evaluation results of the pain threshold; FIG. 3(b) shows the evaluation results of the intestinal permeability of the large intestine (permeability of the large intestine); and FIG. 3(c) shows the relative expression level of Claudin-2. The relative expression level of Claudin-2 is a relative amount when the expression level in group 1 is taken as 100. Each graph shows mean standard error. To analyze a statistically significant difference between the groups, Dunnett test was performed against Group 2 (stress+vehicle group) (*: p<0.05).

The pain threshold was decreased by the stress treatment. The pain threshold was increased by administering the OPC having a galloyl group. The intestinal permeability was increased by the stress treatment, and the OPC having a galloyl group suppressed or ameliorated the increase in the intestinal permeability. The expression level of the tight junction protein Claudin-2 was increased by the stress treatment, and the OPC having a galloyl group suppressed or ameliorated the increase in the expression level. The increase in the expression level of Claudin-2 has been known to cause an increase in intestinal permeability. The increase in the intestinal permeability, and the increase in the expression level of Claudin-2 suggested the decrease in the function of the tight junction in the intestinal epithelial cells due to stress. The OPC having a galloyl group had the action of improving the intestinal barrier function.

### INDUSTRIAL APPLICABILITY

A composition for improving intestinal barrier function according to the present invention is useful in the food or beverage field, the medicine field and the like.

1. A composition for improving intestinal barrier function, the composition comprising:
a compound having a gallic acid residue as an active ingredient,
wherein the compound having a gallic acid residue is at least one compound selected from the group consisting of the following (A1) to (A3):
(A1) a flavan-3-ol polymer having a galloyl group;
(A2) a hydrolyzable tannin; and
(A3) at least one compound selected from the group consisting of catechin gallate, epicatechin gallate, and gallocatechin gallate.

2. The composition for improving intestinal barrier function according to claim 1,
wherein the compound having a gallic acid residue is the flavan-3-ol polymer having a galloyl group.

3. The composition for improving intestinal barrier function according to claim 1,
wherein the flavan-3-ol polymer having a galloyl group is contained in at least one grape-derived raw material selected from the group consisting of grape pulp, grape seed coat, and grape seed.

4. The composition for improving intestinal barrier function according to claim 1,
wherein an amount of gallic acid produced by tannase treatment is 0.1 μg or more per mg of the flavan-3-ol polymer having a galloyl group.

5. The composition for improving intestinal barrier function according to claim 1,
wherein the hydrolyzable tannin is gallotannin or ellagitannin.

6. The composition for improving intestinal barrier function according to claim 5,
wherein the gallotannin is a compound in which 3 to 5 molecules of gallic acid are bound to one molecule of glucose.

7. The composition for improving intestinal barrier function according to claim 5,
wherein the gallotannin is at least one compound selected from the group consisting of 1,4,6-tri-O-galloyl-β-D-glucose, 1,2,4,6-tetra-O-galloyl-β-D-glucose, 1,2,3,6-tetra-O-galloyl-β-D-glucose, 2,3,4,6-tetra-O-galloyl-β-D-glucose, and 1,2,3,4,6-penta-O-galloyl-β-D-glucose.

8. The composition for improving intestinal barrier function according to claim 5,
wherein the ellagitannin is at least one compound selected from the group consisting of tellimagrandin I, pedunculagin, praecoxin A, geraniin, stenophyllanin A, stenophyllanin B, casuarinin, and eugeniflorin D2.

9. The composition for improving intestinal barrier function according to claim 1,
wherein the composition for improving intestinal barrier function is an oral composition.

10. The composition for improving intestinal barrier function according to claim 9,
wherein the oral composition is a food or beverage, a pharmaceutical product, or a quasi-pharmaceutical product.

11. The composition for improving intestinal barrier function according to claim 1,
wherein the composition is used for intestinal regulation by improving the intestinal barrier function.

12. The composition for improving intestinal barrier function according to claim 1,
wherein the composition is used in order to prevent or relieve abdominal discomfort by improving the intestinal barrier function.

13. The composition for improving intestinal barrier function according to claim 1,
wherein the composition is labeled as having an intestinal regulation action.

14. A method for improving intestinal barrier function, the method comprising:
administering at least one compound having a gallic acid residue selected from the group consisting of the following (A1) to (A3) to a subject:
(A1) a flavan-3-ol polymer having a galloyl group;
(A2) a hydrolyzable tannin; and
(A3) at least one compound selected from the group consisting of catechin gallate, epicatechin gallate, and gallocatechin gallate.

15. Use of at least one compound having a gallic acid residue selected from the group consisting of the following (A1) to (A3) for improving intestinal barrier function:
(A1) a flavan-3-ol polymer having a galloyl group;
(A2) a hydrolyzable tannin; and
(A3) at least one compound selected from the group consisting of catechin gallate, epicatechin gallate, and gallocatechin gallate.
